(19) United States
(12) Patent Application Publication
(10) Pub. No.: US 2025/0128067 A1
(43) Pub. Date: Apr. 24, 2025

(54) CONTEXT-DEPENDENT WEIGHTING FOR CLOSED-LOOP NEUROMODULATION TREATMENT

(71) Applicant: Boston Scientific Neuromodulation Corporation, Valencia, CA (US)

(72) Inventor: Bradley Lawrence Hershey, Carrollton, TX (US)

(21) Appl. No.: 18/903,886

(22) Filed: Oct. 1, 2024

Related U.S. Application Data
(60) Provisional application No. 63/545,407, filed on Oct. 24, 2023.

(51) Int. Cl. A61N 1/36; G16H 10/60; G16H 20/40; G16H 40/63

(52) U.S. Cl. CPC A61N 1/36139; A61N 1/36178; G16H 20/40; G16H 40/63; G16H 10/60

(57) ABSTRACT

Systems and techniques are disclosed to adapt neurostimulation programming in closed-loop adjustment of an implantable neurostimulation device, based on evaluation and weighting of patient data that excludes or reduces effects of interfering events. In an example, adapting a neurostimulation programming model includes: obtaining patient data observed during a prior time period; identifying events experienced by the patient during the prior time period (e.g., confounding events, intervention events) that cause variance in measurements of patient data; determining weighted data by weighting patient data observed during the one or more events, as the weighting of the patient data reduces effects of the patient data during the one or more events on a modification of the programming model; and modifying (e.g., re-training, reinforcing, or adapting) the programming model based on the weighted data. The modified programming model can then be used to generate updated programming parameters for subsequent neurostimulation treatment.

### CLAIM OF PRIORITY

This application claims the benefit of U.S. Provisional Application No. 63/545,407, filed on Oct. 24, 2023, which is hereby incorporated by reference in its entirety.

### TECHNICAL FIELD

The present disclosure relates generally to medical devices, and more particularly, to systems, devices, and methods for electrical stimulation programming techniques using artificial intelligence models and related mechanisms for closed loop programming, to control implanted electrical stimulation for treatment and/or management of medical conditions.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system.

A neurostimulation system can be used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to produce an analgesic effect that masks pain sensation, or to produce a functional effect that allows increased movement or activity of the patient. Other forms of neurostimulation may include a DBS system that uses similar pulses of electricity at particular locations in the brain to reduce symptoms of essential tremors, Parkinson's disease, psychological disorders, or the like.

While modern electronics can accommodate the need for generating and delivering neurostimulation energy in a variety of forms, the capability of a neurostimulation system depends on its post-manufacturing programmability to a great extent. Some techniques have been developed for automated or closed-loop programming, which involves collecting and analyzing a variety of patient data to drive programming recommendations and modifications. However, it is often not clear whether the changes in patient data are attributable to use of the neurostimulation, a change in the medical condition being treated, or another factor. As a result, closed-loop programming may not provide accurate programming changes in some scenarios.

### SUMMARY

The following Summary provides examples as an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

Example 1 is a system for adapting a neurostimulation programming model used for neurostimulation treatment, the system comprising: at least one processor; and at least one memory device comprising instructions, which when executed by the processor, cause the processor to perform operations that: obtain patient data observed during a prior time period, the patient data for a human patient having a neurostimulation treatment delivered with a neurostimulation device to treat a medical condition; identify one or more events experienced by the human patient during the prior time period that cause variance in measurements of the patient data; determine weighted data by weighting the patient data observed during the one or more events; and modify the neurostimulation programming model based on the weighted data, the neurostimulation programming model to generate updated programming parameters for the neurostimulation treatment, wherein the weighting of the patient data reduces effects of the patient data during the one or more events on the neurostimulation programming model.

In Example 2, the subject matter of Example 1 optionally includes subject matter where the one or more events include an intervention event that is related to treatment of the medical condition of the human patient, and wherein to determine the weighted data for the intervention event includes to calculate a weight that reduces the effects of the patient data throughout a duration of the intervention event.

In Example 3, the subject matter of any one or more of Examples 1-2 optionally include subject matter where the one or more events include a confounding event that is not related to treatment of the medical condition of the human patient, and wherein to determine the weighted data for the confounding event includes to calculate a weight that reduces the effects of the patient data throughout a duration of the confounding event.

In Example 4, the subject matter of any one or more of Examples 1-3 optionally include the processor further to perform operations that: calculate weights for the patient data throughout the prior time period for respective measurements of the patient data, wherein the calculation of the weights uses a discount that reduces effects of the respective measurements on the neurostimulation programming model, based on an amount of time elapsed.

In Example 5, the subject matter of Example 4 optionally includes subject matter where to calculate the weights for the patient data throughout the prior time period for the respective measurements of the patient data, includes to calculate the weights for the patient data based on a rate of decrease in data relevance and a maximum value of the data relevance.

In Example 6, the subject matter of any one or more of Examples 1-5 optionally include subject matter where to determine the weighted data corresponding to a respective event of the one or more events, includes to calculate a weight based on a duration for the respective event.

In Example 7, the subject matter of any one or more of Examples 1-6 optionally include subject matter where to determine the weighted data corresponding to a respective event of the one or more events, includes to calculate a weight based on an estimated impact of the respective event on data relevance, and wherein the estimated impact differs based on a type or severity of the respective event.

In Example 8, the subject matter of any one or more of Examples 1-7 optionally include subject matter where to modify the neurostimulation programming model includes to perform reinforcement or re-training of the neurostimulation programming model using the weighted data.

In Example 9, the subject matter of any one or more of Examples 1-8 optionally include subject matter where the neurostimulation programming model is implemented as an artificial neural network or as a machine learning classifier.

In Example 10, the subject matter of any one or more of Examples 1-9 optionally include the processor further to perform operations that: extract the patient data from a patient data source, wherein the patient data source provides at least one of: freeform text, voice recordings, survey data, or medical records data.

In Example 11, the subject matter of any one or more of Examples 1-10 optionally include the processor further to perform operations that: identify, with the use of the neurostimulation programming model, programming parameters for use with the neurostimulation device.

In Example 12, the subject matter of Example 11 optionally includes the processor further to perform operations that: communicate, to the neurostimulation device, at least one command to cause the use of the identified programming parameters.

In Example 13, the subject matter of Example 12 optionally includes subject matter where the identified programming parameters specify operation of a neurostimulation program including one or more of: pulse patterns, pulse shapes, a spatial location of pulses, waveform shapes, or a spatial location of waveform shapes, for modulated energy provided with a plurality of leads of the neurostimulation device.

Example 14 is a machine-readable medium including instructions, which when executed by a machine, cause the machine to perform the operations of the system of any of the Examples 1 to 13.

Example 15 is a method to perform the operations of the system of any of the Examples 1 to 13.

Example 16 is a device for adapting a neurostimulation programming model used for neurostimulation treatment, the device comprising: at least one processor and at least one memory; event data processing circuitry, operably coupled with the processor and the memory, the event data processing circuitry configured to: obtain patient data observed during a prior time period, the patient data for a human patient having a neurostimulation treatment delivered with a neurostimulation device to treat a medical condition; identify one or more events experienced by the human patient during the prior time period that cause variance in measurements of the patient data; determine weighted data by weighting the patient data observed during the one or more events; and neurostimulation programming circuitry, operably coupled with the at least one processor and the at least one memory, the neurostimulation programming circuitry configured to: modify the neurostimulation programming model based on the weighted data, the neurostimulation programming model to generate updated programming parameters for the neurostimulation treatment, wherein the weighting of the patient data reduces effects of the patient data during the one or more events on the neurostimulation programming model.

In Example 17, the subject matter of Example 16 optionally includes subject matter where the one or more events include an intervention event that is related to treatment of the medical condition of the human patient, and wherein to determine the weighted data for the intervention event includes to calculate a weight that reduces the effects of the patient data throughout a duration of the intervention event.

In Example 18, the subject matter of any one or more of Examples 16-17 optionally include subject matter where the one or more events include a confounding event that is not related to treatment of the medical condition of the human patient, and wherein to determine the weighted data for the confounding event includes to calculate a weight that reduces the effects of the patient data throughout a duration of the confounding event.

In Example 19, the subject matter of any one or more of Examples 16-18 optionally include the event data processing circuitry further configured to: calculate weights for the patient data throughout the prior time period for respective measurements of the patient data, wherein the calculation of the weights uses a discount that reduces effects of the respective measurements on the neurostimulation programming model, based on an amount of time elapsed.

In Example 20, the subject matter of Example 19 optionally includes subject matter where to calculate the weights for the patient data throughout the prior time period for the respective measurements of the patient data, includes to calculate the weights for the patient data based on a rate of decrease in data relevance and a maximum value of the data relevance.

In Example 21, the subject matter of any one or more of Examples 16-20 optionally include subject matter where to determine the weighted data corresponding to a respective event of the one or more events, includes to calculate a weight based on a duration for the respective event.

In Example 22, the subject matter of any one or more of Examples 16-21 optionally include subject matter where to determine the weighted data corresponding to a respective event of the one or more events, includes to calculate a weight based on an estimated impact of the respective event on data relevance, and wherein the estimated impact differs based on a type or severity of the respective event.

In Example 23, the subject matter of any one or more of Examples 16-22 optionally include subject matter where to modify the neurostimulation programming model includes to perform reinforcement or re-training of the neurostimulation programming model using the weighted data, and wherein the neurostimulation programming model is implemented as an artificial neural network or as a machine learning classifier.

In Example 24, the subject matter of any one or more of Examples 16-23 optionally include the neurostimulation programming circuitry further configured to: identify, with the use of the neurostimulation programming model, programming parameters for use with the neurostimulation device; and communicate, to the neurostimulation device, at least one command to cause the use of the identified programming parameters.

In Example 25, the subject matter of Example 24 optionally includes subject matter where the identified programming parameters specify operation of a neurostimulation program including one or more of: pulse patterns, pulse shapes, a spatial location of pulses, waveform shapes, or a spatial location of waveform shapes, for modulated energy provided with a plurality of leads of the neurostimulation device.

Example 26 is a method for use to adapt a neurostimulation programming model used for neurostimulation treatment, the method comprising a plurality of operations executed with at least one processor of a computing device, the plurality of operations comprising: obtaining patient data observed during a prior time period, the patient data for a human patient having a neurostimulation treatment delivered with a neurostimulation device to treat a medical condition; identifying one or more events experienced by the human patient during the prior time period that cause variance in measurements of the patient data; determining weighted data by weighting the patient data observed during the one or more events; and modifying the neurostimulation programming model based on the weighted data, the neurostimulation programming model to generate updated programming parameters for the neurostimulation treatment, wherein the weighting of the patient data reduces effects of the patient data during the one or more events on the neurostimulation programming model.

In Example 27, the subject matter of Example 26 optionally includes subject matter where the one or more events include an intervention event that is related to treatment of the medical condition of the human patient, and wherein to determine the weighted data for the intervention event includes to calculate a weight that reduces the effects of the patient data throughout a duration of the intervention event.

In Example 28, the subject matter of any one or more of Examples 26-27 optionally include subject matter where the one or more events include a confounding event that is not related to treatment of the medical condition of the human patient, and wherein to determine the weighted data for the confounding event includes to calculate a weight that reduces the effects of the patient data throughout a duration of the confounding event.

In Example 29, the subject matter of any one or more of Examples 26-28 optionally include the operations further comprising: calculating weights for the patient data throughout the prior time period for respective measurements of the patient data, wherein the calculation of the weights uses a discount that reduces effects of the respective measurements on the neurostimulation programming model, based on an amount of time elapsed.

In Example 30, the subject matter of Example 29 optionally includes subject matter where calculating the weights for the patient data throughout the prior time period for the respective measurements of the patient data, includes calculating the weights for the patient data based on a rate of decrease in data relevance and a maximum value of the data relevance.

In Example 31, the subject matter of any one or more of Examples 26-30 optionally include subject matter where determining the weighted data corresponding to a respective event of the one or more events, includes calculating a weight based on a duration for the respective event.

In Example 32, the subject matter of any one or more of Examples 26-31 optionally include subject matter where determining the weighted data corresponding to a respective event of the one or more events, includes calculating a weight based on an estimated impact of the respective event on data relevance, and wherein the estimated impact differs based on a type or severity of the respective event.

In Example 33, the subject matter of any one or more of Examples 26-32 optionally include subject matter where modifying the neurostimulation programming model includes performing reinforcement or re-training of the neurostimulation programming model using the weighted data, and wherein the neurostimulation programming model is implemented as an artificial neural network or as a machine learning classifier.

In Example 34, the subject matter of any one or more of Examples 26-33 optionally include the operations further comprising: identifying, with the use of the neurostimulation programming model, programming parameters for use with the neurostimulation device; and communicating, to the neurostimulation device, at least one command to cause the use of the identified programming parameters.

In Example 35, the subject matter of Example 34 optionally includes subject matter where the identified programming parameters specify operation of a neurostimulation program including one or more of: pulse patterns, pulse shapes, a spatial location of pulses, waveform shapes, or a spatial location of waveform shapes, for modulated energy provided with a plurality of leads of the neurostimulation device.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example, an embodiment of a neurostimulation system.

FIG. 2 illustrates, by way of example, an embodiment of a stimulation device and a lead system, such as may be implemented in the neurostimulation system of FIG. 1.

FIG. 3 illustrates, by way of example, an embodiment of a programming device, such as may be implemented in the neurostimulation system of FIG. 1.

FIG. 4 illustrates, by way of example, an implantable neurostimulation system and portions of an environment in which the system may be used.

FIG. 5 illustrates, by way of example, an embodiment of an implantable stimulator and one or more leads of a neurostimulation system, such as the implantable neurostimulation system of FIG. 4.

FIG. 6 illustrates, by way of example, an embodiment of a closed-loop programming system used with a neurostimulation system, such as the implantable neurostimulation system of FIG. 4.

FIG. 7 illustrates, by way of example, an embodiment of data interactions among a closed-loop programming device, a program modeling system, and physician and patient interaction computing devices, for closed-loop operation of a neurostimulation device based on context-dependent weighting.

FIG. 8 illustrates, by way of example, an embodiment of data weighting used for training an artificial intelligence model, adapted for closed-loop operation of a neurostimulation device.

FIGS. 9A and 9B illustrate, by way of example, graphs that demonstrate a coefficient of reliability versus time, used for the analysis of events in connection with an embodiment for closed-loop operation of a neurostimulation device.

FIG. 10 illustrates, by way of example, a data flow for detecting and analyzing events, in connection with an embodiment for closed-loop operation of a neurostimulation device.

FIG. 11 illustrates, by way of example, a closed-loop processing flow implementing neurostimulation treatment of a human patient, using context-dependent weighting for training data in a neurostimulation programming model.

FIG. 12 illustrates, by way of example, a flowchart of a method implemented by a system or device to adapt a neurostimulation programming model used for neurostimulation treatment.

FIG. 13 illustrates, by way of example, a block diagram of an embodiment of a computing system implementing data weighting and data processing to modify operation of a closed-loop therapy model used for neurostimulation programming.

FIG. 14 illustrates, by way of example, a block diagram of an embodiment of a computing system implementing neurostimulation programming circuitry, to cause programming of an implantable electrical neurostimulation device.

FIG. 15 is a block diagram illustrating a machine in the example form of a computer system, within which a set or sequence of instructions may be executed to cause the machine to perform any one of the methodologies discussed herein, according to an example embodiment.

### DETAILED DESCRIPTION

This document discusses a neurostimulation therapy model adapted to determine or recommend programming values for implantable electrical neurostimulation device, for use in the treatment of pain or similar physiological conditions in a human subject (e.g., a patient). This document also discusses approaches for improving the tuning or re-training of this therapy model, based on a context-dependent weighting algorithm or process that filters and weighs certain types of patient-specific data during the tuning or re-training of the therapy model.

Closed-loop neurostimulation programming generally refers to a fully or partially automated use of a therapy model that recommends (or automatically causes) changes to the operation of a neurostimulation or neuromodulation device. Closed-loop therapy models can be trained using historical outcomes (e.g., pain, sleep, activity, mood, etc.) and paired neurostimulation device usage (e.g., program, amplitude) collected from a variety of digital health data sources. These data sources may include personal devices such as smartphones, which enable patients to provide detailed feedback information (e.g., freeform text information, voice recordings or captured audio data, survey answers, etc.) about the patient's state in an at-home setting, including detailed information related to the patient state and whether the treatment is or is not working. After training the therapy model, the model can recommend neurostimulation settings (e.g., programming parameters, program usage) that optimize a clinically-relevant goal for the treatment (e.g., to minimize pain intensity, minimize distance to best patient state, etc.). However, not all patient data is of equal value for training or tuning (optimizing) a therapy model, causing some closed-loop (or partially-closed-loop) neurostimulation treatments to recommend programs or settings that are not a best fit for therapy objectives.

Older data may have lower reliability because it is more likely that previously applied settings may no longer be ideal, due to changes caused by the dynamic nature of chronic pain and the likelihood of lead migration. Likewise, data collected during certain events can be less reliable—and, less relevant for use in modifying, training, or adjusting a model-due to the reduced validity of attributing the measured outcomes to the neurostimulation therapy. This includes data collected during medical treatment events that involve some medical condition intervention, and data collected during events that have confounding impacts such as an unrelated sickness or emotional event. The present techniques and systems improve these scenarios by applying weighting to reduce, discount, or eliminate the impact of unreliable data from certain events on the training of the programming model.

As an example, various systems and methods are described to generate, identify, implement, or adjust parameters of neurostimulation treatment in a closed-loop therapy approach. These parameters may be generated after model re-training and modification based on patient-specific data weighting, to reduce or discount the impact of confounding or intervention events experienced by the patient. Additional details are described to operate an algorithm that calculates or adjusts the amount of patient data weighting for events based on elapsed time, and to operate an algorithm that detects or classifies specific types of events that can impact data relevance.

The model re-training and modification may be implemented in a variety of types of therapy models in a closed-loop programming system or other neurostimulation programming implementation (e.g., algorithm, model, rule set, etc.) that evaluates different types and amounts of patient-specified therapy objectives. In various examples, the model re-training and modification may be integrated with existing programming workflows or operations of intelligent or closed-loop neuromodulation programming systems, including those implementing aspects of artificial intelligence (AI), such as machine learning, neural networks, decision trees, and the like. Thus, the model re-training and modification approaches may be used in a variety of implementations that pre-process data considered by closed-loop programming, and are not limited to any specific type of neural network or AI model.

The following document also describes ways in which a therapy model can be used in a closed-loop setting to generate, identify, modify, select, or recommend operational parameters of the neurostimulation device. By way of example, such operational parameters may include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The neurostimulator may use current or voltage sources to provide the neurostimulator output, and apply any number of control techniques to modify the electrical stimulation applied to anatomical sites or systems related to pain or analgesic effect. In various embodiments, a neurostimulator program may be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

In various embodiments, the present subject matter may be implemented using a combination of hardware and software designed to collect patient state information from users, including feedback information from patients directly, or information directly or indirectly provided by caregivers, clinicians, or researchers. Other aspects of the hardware and software may connect with medical information systems to determine historical events from at least one prior time period, and to apply specific types of rules for the evaluation of the historical events. Other combinations of hardware and software including cloud-based programming may be used to process the patient state information, and apply weighting or apply training from the various types of events detected from the patient state information. Finally, other combinations of hardware and software including end-user software apps may be used to generate, identify, select, implement, and update neurostimulation programs that achieve the therapy objectives, using a therapy model that has been modified or adapted based on the weighting.

The implementation of neurostimulation programs, particularly in a closed-loop system, may result in variation in the location, intensity, and type of defined waveforms and patterns in an effort to increase therapeutic efficacy and/or patient satisfaction for neurostimulation therapies, such as SCS and DBS therapies. While neurostimulation is specifically discussed as an example, the present subject matter may apply to other therapies that employ stimulation pulses of electrical or other forms of energy for treating chronic pain or like physiological or psychological conditions. Additionally, the delivery of neurostimulation energy that is discussed herein may be delivered in the form of electrical neurostimulation pulses. The delivery may be controlled using stimulation parameters that specify spatial (where to stimulate), temporal (when to stimulate), and informational (patterns of pulses directing the nervous system to respond as desired) aspects of a pattern of neurostimulation pulses.

Many current neurostimulation systems are programmed to deliver periodic pulses with one or a few uniform waveforms continuously or in bursts. However, neural signals may include more sophisticated patterns to communicate various types of information, including sensations of pain, pressure, temperature, etc. Accordingly, the following drawings provide an introduction to the features of an example neurostimulation system and how programming of a neurostimulation system may be accomplished through open-loop or closed-loop neurostimulation systems.

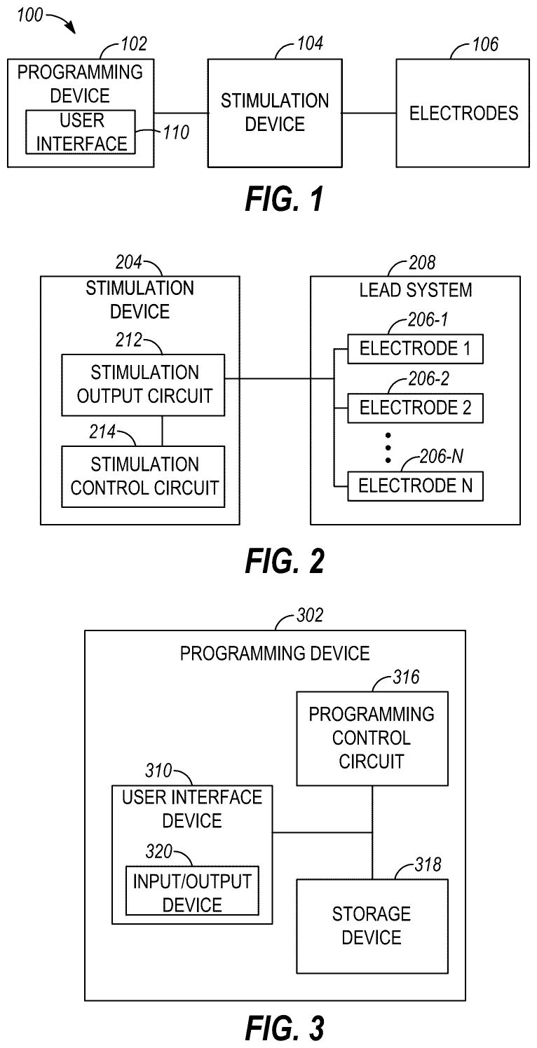

FIG. 1 illustrates an embodiment of a neurostimulation system 100. System 100 includes electrodes 106, a stimulation device 104, and a programming device 102. Electrodes 106 are configured to be placed on or near one or more neural targets in a patient. Stimulation device 104 is configured to be electrically connected to electrodes 106 and deliver neurostimulation energy, such as in the form of electrical pulses, to the one or more neural targets through electrodes 106. The delivery of the neurostimulation is controlled by using a plurality of stimulation parameters, such as stimulation parameters specifying a pattern of the electrical pulses and a selection of electrodes through which each of the electrical pulses is delivered. In various embodiments, at least some parameters of the plurality of stimulation parameters are selected or programmable by a clinical user, such as a physician or other caregiver who treats the patient using system 100; however, some or all of the parameters may also be provided in connection with closed-loop programming logic and adjustment. Programming device 102 provides the user with accessibility to implement, change, or modify the programmable parameters, and to provide information related to patient state or treatment controls. In various embodiments, programming device 102 is configured to be communicatively coupled to stimulation device 104 via a wired or wireless link.

In various embodiments, programming device 102 includes a user interface 110 (e.g., a user interface embodied by a graphical, text, voice, or hardware-based user interface) that allows the user to set and/or adjust values of the user-programmable parameters by creating, editing, loading, and removing programs that include parameter combinations such as patterns and waveforms. These adjustments may also include changing and editing values for the user-programmable parameters or sets of the user-programmable parameters individually (including values set in response to a therapy efficacy indication). Such waveforms may include, for example, the waveform of a pattern of neurostimulation pulses to be delivered to the patient as well as individual waveforms that are used as building blocks of the pattern of neurostimulation pulses. Examples of such individual waveforms include pulses, pulse groups, and groups of pulse groups. The program and respective sets of parameters may also define an electrode selection specific to each individually defined waveform.

As described in more detail below, a user, e.g., the patient, a caregiver, or a clinician or other medical professional associated with the patient, can provide inputs that are used to train or adapt a closed-loop programming model or logic based on patient state information and patient data history events. This closed-loop programming model or logic is then used to select, load, modify, and implement one or more parameters of a defined program for neurostimulation treatment so that the neurostimulation treatment can correctly include or exclude the consideration of certain history events. The closed-loop programming model or logic then may identify or recommend a variety of programming parameters, programs, or programming settings, including a program or parameter change within a program that is likely to produce an improvement for the treatment objectives (such as to address pain, mobility, sleep disruption, and the like). Example parameters that can be implemented by a selected neurostimulation program include, but are not limited to the following: amplitude, pulse width, frequency, duration, total charge injected per unit time, cycling (e.g., on/off time), pulse shape, number of phases, phase order, interphase time, charge balance, ramping, as well as spatial variance (e.g., electrode configuration changes over time).

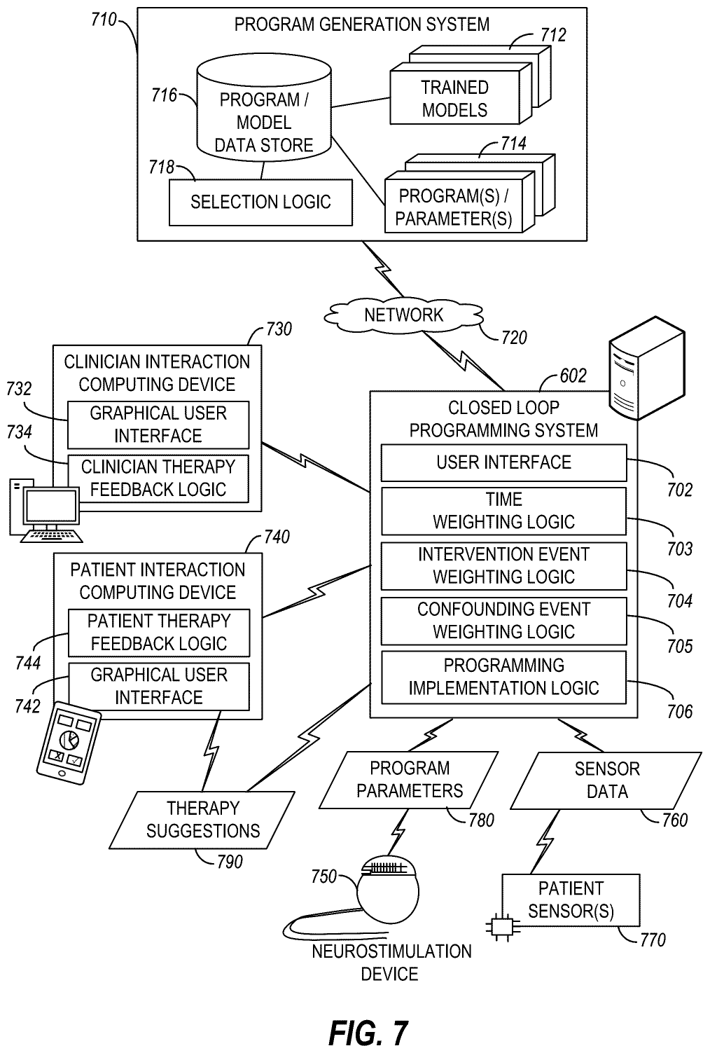

As detailed in FIG. 6, a controller, e.g., controller 630 of FIG. 6, can implement program(s) and parameter setting(s) to affect a specific neurostimulation waveform, pattern, or energy output, using a program or setting in storage, e.g., external storage device 616 of FIG. 6, or using settings communicated via an external communication device 618 of FIG. 6 corresponding to the selected program. The implementation of such program(s) or setting(s) may further define a therapy strength and treatment type corresponding to a specific pulse group, or a specific group of pulse groups, based on the specific program(s) or setting(s). As also described in more detail below with respect to FIG. 7 and thereafter, a program modeling system and closed-loop programming system may operate to identify, select, produce, or generate this information in closed-loop feedback configuration, as user input and physiological sensor data is observed and monitored for further refinement. In addition to the use of closed-loop feedback, other forms of input from a clinician, a caregiver, or the patient may also affect use and implementation of selected parameters or programs of the program modeling system and closed-loop programming system, including in settings where a combination of dynamic (automatic) and manual control are involved.

Portions of the stimulation device 104, e.g., implantable medical device, or the programming device 102 can be implemented using hardware, software, or any combination of hardware and software. Portions of the stimulation device 104 or the programming device 102 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. The system 100 could also include a subcutaneous medical device (e.g., subcutaneous ICD, subcutaneous diagnostic device), wearable medical devices (e.g., patch-based sensing device), or other external medical devices.

FIG. 2 illustrates an embodiment of a stimulation device 204 and a lead system 208, such as may be implemented in neurostimulation system 100 of FIG. 1. Stimulation device 204 represents an embodiment of stimulation device 104 and includes a stimulation output circuit 212 and a stimulation control circuit 214. Stimulation output circuit 212 produces and delivers neurostimulation pulses, including the neurostimulation waveform and parameter settings implemented via a program selected or implemented with the user interface 110. Stimulation control circuit 214 controls the delivery of the neurostimulation pulses using the plurality of stimulation parameters, which specifies a pattern of the neurostimulation pulses. Lead system 208 includes one or more leads each configured to be electrically connected to stimulation device 204 and a plurality of electrodes 206 distributed in the one or more leads. The plurality of electrodes 206 includes electrode 206-1, electrode 206-2, . . . electrode 206-N, each a single electrically conductive contact providing for an electrical interface between stimulation output circuit 212 and tissue of the patient, where N≥2. The neurostimulation pulses are each delivered from stimulation output circuit 212 through a set of electrodes selected from electrodes 206. In various embodiments, the neurostimulation pulses may include one or more individually defined pulses, and the set of electrodes may be individually definable by the user for each of the individually defined pulses.

In various embodiments, the number of leads and the number of electrodes on each lead depend on, for example, the distribution of target(s) of the neurostimulation and the need for controlling the distribution of an electric field at each target. In one embodiment, lead system 208 includes 2 leads each having 8 electrodes. Those of ordinary skill in the art will understand that the neurostimulation system 100 may include additional components such as sensing circuitry for patient monitoring and/or feedback control of the therapy, telemetry circuitry, and power. The neurostimulation system 100 may also integrate with other sensors, or such other sensors may independently provide information for use with programming of the neurostimulation system 100.

The neurostimulation system may be configured to modulate spinal target tissue or other neural tissue. The configuration of electrodes used to deliver electrical pulses to the targeted tissue constitutes an electrode configuration, with the electrodes capable of being selectively programmed to act as anodes (positive), cathodes (negative), or left off (zero). In other words, an electrode configuration represents the polarity being positive, negative, or zero. Other parameters that may be controlled or varied include the amplitude, pulse width, and rate (or frequency) of the electrical pulses. Each electrode configuration, along with the electrical pulse parameters, can be referred to as a “modulation parameter” set. Each set of modulation parameters, including fractionalized current distribution to the electrodes (as percentage cathodic current, percentage anodic current, or off), may be stored and combined into a program that can then be used to modulate multiple regions within the patient.

The neurostimulation system may be configured to deliver different electrical fields to achieve a temporal summation of modulation. The electrical fields can be generated respectively on a pulse-by-pulse basis. For example, a first electrical field can be generated by the electrodes (using a first current fractionalization) during a first electrical pulse of the pulsed waveform, a second different electrical field can be generated by the electrodes (using a second different current fractionalization) during a second electrical pulse of the pulsed waveform, a third different electrical field can be generated by the electrodes (using a third different current fractionalization) during a third electrical pulse of the pulsed waveform, a fourth different electrical field can be generated by the electrodes (using a fourth different current fractionalized) during a fourth electrical pulse of the pulsed waveform, and so forth. These electrical fields can be rotated or cycled through multiple times under a timing scheme, where each field is implemented using a timing channel. The electrical fields may be generated at a continuous pulse rate, or as bursts of pulses. Furthermore, the interpulse interval (i.e., the time between adjacent pulses), pulse amplitude, and pulse duration during the electrical field cycles may be uniform or may vary within the electrical field cycle. Some examples are configured to determine a modulation parameter set to create a field shape to provide a broad and uniform modulation field such as may be useful to prime targeted neural tissue with sub-perception modulation. Some examples are configured to determine a modulation parameter set to create a field shape to reduce or minimize modulation of non-targeted tissue (e.g., dorsal column tissue). Various examples disclosed herein are directed to shaping the modulation field to enhance modulation of some neural structures and diminish modulation at other neural structures. The modulation field may be shaped by using multiple independent current control (MICC) or multiple independent voltage control to guide the estimate of current fractionalization among multiple electrodes and estimate a total amplitude that provide a desired strength. For example, the modulation field may be shaped to enhance the modulation of dorsal horn neural tissue and to minimize the modulation of dorsal column tissue. A benefit of MICC is that MICC accounts for various in electrode-tissue coupling efficiency and perception threshold at each individual contact, so that “hotspot” stimulation is eliminated.

The number of electrodes available combined with the ability to generate a variety of complex electrical pulses, presents a huge selection of available modulation parameter sets to the clinician or patient. For example, if the neurostimulation system to be programmed has sixteen electrodes, millions of modulation parameter value combinations may be available for programming into the neurostimulation system. Furthermore, some SCS systems have as many as thirty-two electrodes, which exponentially increases the number of modulation parameter value combinations available for programming. The implementation and use of a closed-loop programming system and program modeling system as described further in FIGS. 6 to 11 and thereafter provides a mechanism for identifying and recommending programs and program parameters in a closed-loop fashion that increases the effects of data collected during certain prior time periods and events (while reducing the effects of certain types of intervention events or confounding events).

FIG. 3 illustrates an embodiment of a programming device 302, such as may be implemented in neurostimulation system 100. Programming device 302 represents an embodiment of programming device 102 and includes a storage device 318, a programming control circuit 316, and a user interface device 310. Programming control circuit 316 generates the plurality of stimulation parameters that controls the delivery of the neurostimulation pulses according to the pattern of the neurostimulation pulses. The user interface device 310 represents an embodiment to implement the user interface 110.

In various embodiments, the user interface device 310 includes an input/output device 320 that is capable to receive user interaction and commands to load, modify, and implement neurostimulation programs and schedule delivery of the neurostimulation programs. In various embodiments, the input/output device 320 allows the user to create, establish, access, and implement respective parameter values of a neurostimulation program through graphical selection (e.g., in a graphical user interface output with the input/output device 320), or other graphical input/output relating to therapy objectives, efficacy of applied treatment, user feedback, event information, and the like. In various examples, the user interface device 310 can receive user input to initiate or control the implementation of the programs or program changes that are recommended, modified, selected, or loaded through use of a closed-loop programming system, described in more detail below. Other input and output related to particular types of events and patient state may also be exchanged via the input/output device 320.

In various embodiments, the input/output device 320 allows the patient user to apply, change, modify, or discontinue certain building blocks of a program and a frequency at which a selected program is delivered, such as based on recommendations provided from a closed-loop programming system. In various embodiments, the input/output device 320 can allow the patient user to save, retrieve, and modify programs (and program settings) loaded from a clinical encounter, managed from the patient feedback computing device, or stored in storage device 318 as templates. In various embodiments, the input/output device 320 and accompanying software on the user interface device 310 allows newly created building blocks, program components, programs, and program modifications to be saved, stored, or otherwise persisted in storage device 318. Thus, it will be understood that the user interface device 310 may allow many forms of device operation and control, even as closed-loop programming is occurring.

In one embodiment, the input/output device 320 includes a touchscreen. In various embodiments, the input/output device 320 includes any type of presentation device, such as interactive or non-interactive screens, and any type of user input device that allows the user to interact with a user interface to implement, remove, or schedule the programs. Thus, the input/output device 320 may include one or more of a touchscreen, keyboard, keypad, touchpad, trackball, joystick, and mouse. The logic of the user interface 110, the stimulation control circuit 214, and the programming control circuit 316, including their various embodiments discussed in this document, may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

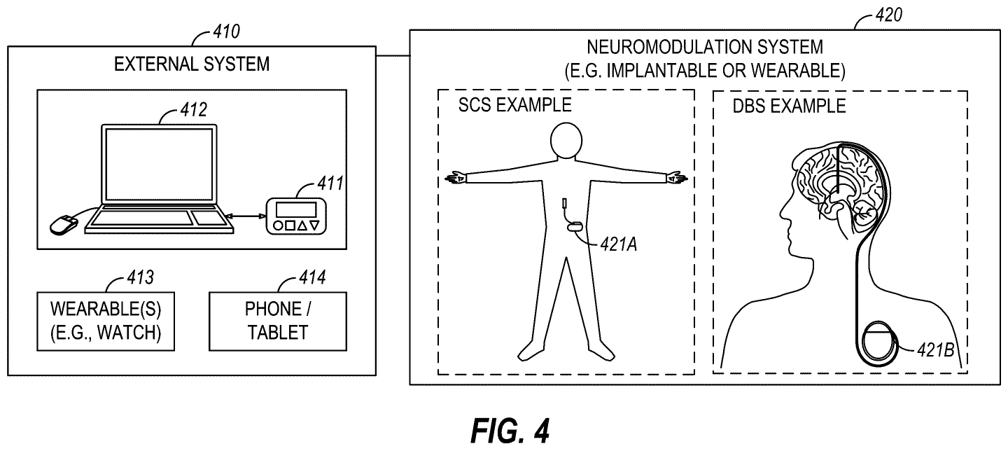

FIG. 4 illustrates an implantable neurostimulation system 420 and portions of an environment in which system 420 may be used, such as may be implemented as the stimulation device 104, 204 illustrated in FIGS. 1 and 2. FIG. 4 specifically illustrates, by way of example and not limitation, the neurostimulation system 100 of FIG. 1 implemented in a spinal cord stimulation (SCS) system or a deep brain stimulation (DBS) system. The illustrated neuromodulation system 420 connects with an external system 410 that may include at least one programming device. The illustrated external system 410 may include a programmer 412 (e.g., clinician programmer) configured for use by a clinician to communicate with and program the neurostimulator, and a remote control device 411 configured for use by the patient to communicate with and program the neurostimulator. For example, the remote control device 411 may allow the patient to turn a therapy on and off and/or may allow the patient to adjust patient-programmable parameter(s) of the plurality of modulation parameters (e.g., by switching programs).

FIG. 4 further illustrates the neuromodulation system 420 as an ambulatory medical device, such as implemented by stimulation device 421A or stimulation device 421B. Examples of ambulatory devices include wearable or implantable neuromodulators. The external system 410 may include a network of computers, including computer(s) remotely located from the ambulatory medical device that are capable of communicating via one or more communication networks with the programmer 412 and/or the remote control device 411. The remotely located computer(s) and the ambulatory medical device may be configured to communicate with each other via another external device such as the programmer 412 or the remote control device 411.

The external system 410 may also include one or more wearables 413 and a portable device 414 such as a smartphone or tablet. In some examples, the wearables 413 and the portable device 414 may allow a user to obtain and provide input data, such as sensor data values (e.g., from a physiologic sensor of a wearable) or feedback/status information (e.g., on a phone/tablet screen) in connection with a data collection process. In some examples, the remote control device 411 and/or the programmer 412 also may display recommendations or program settings (e.g., derived from recommendations as part of a fully or partially closed-loop programming process). The remote control device 411 and/or the programmer 412 may be used to communicate other aspects of input and output, including inputs from the usage data of various neurostimulation programs, events associated with such programs, and the like.

In various embodiments, the external system 410 includes one or more external (non-implantable) devices or interfaces, each allowing the user and/or the patient to communicate with the implantable system 420. In some embodiments, the external system 410 includes a programming device intended for the user to initialize and adjust settings for the implantable stimulator 421 and a remote control device intended for use by the patient. For example, the remote control device 411 may allow the patient to turn the implantable stimulator 421 on and off and/or adjust certain patient-programmable parameters of the plurality of stimulation parameters. The remote control device 411, the portable device 414, or the wearable 413 may also provide a mechanism to receive and process feedback on the operation of the implantable neuromodulation system. Feedback may include metrics or an efficacy indication reflecting perceived pain, effectiveness of therapies, or other aspects of patient comfort or condition. Feedback may also include information relating to events in the patient's life, including event information that is not directly related to (e.g., caused by) the medical condition or symptoms being treated. This feedback may be used to identify whether an event is a confounding or a medical interventional event, or another type of event. Feedback may be automatically detected from a patient's physiological state, collected from other sensors or devices (not shown), or manually obtained from user input (e.g., freeform text, voice or audio recordings, survey answers, user input selections) entered in a user interface.

As used herein, the terms “neurostimulator,” “stimulator,” “neurostimulation,” and “stimulation” generally refer to the delivery of electrical energy that affects the neuronal activity of neural tissue, which may be excitatory or inhibitory; for example by initiating an action potential, inhibiting or blocking the propagation of action potentials, affecting changes in neurotransmitter/neuromodulator release or uptake, and inducing changes in neuro-plasticity or neurogenesis of tissue. It will be understood that other clinical effects and physiological mechanisms may also be provided through use of such stimulation techniques.

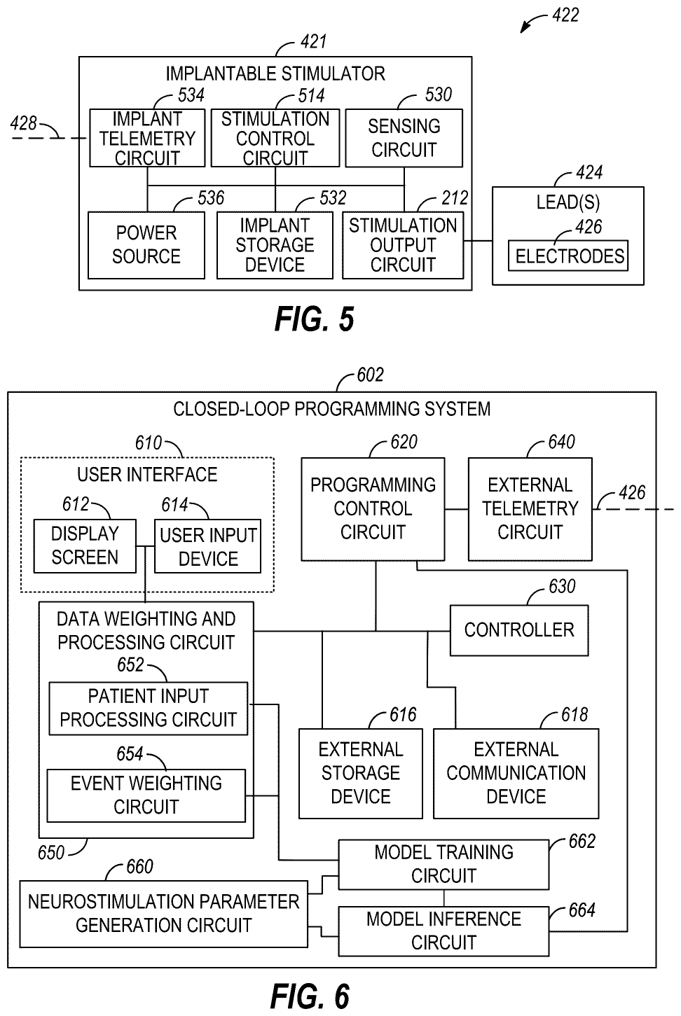

FIG. 5 illustrates an embodiment of the implantable stimulator 421 and the one or more leads 424 of an implantable neurostimulation system, such as the implantable system 420. The implantable stimulator 421 may include a sensing circuit 530 used for an optional sensing capability, stimulation output circuit 212, a stimulation control circuit 514, an implant storage device 532, an implant telemetry circuit 534, and a power source 536. The sensing circuit 530, when included and needed, senses one or more physiological signals for purposes of patient monitoring and/or feedback control of the neurostimulation, including in the closed-loop programming processes discussed herein. Examples of the one or more physiological signals includes neural and other signals each indicative of a condition of the patient that is treated by the neurostimulation and/or a response of the patient to the delivery of the neurostimulation.

The stimulation output circuit 212 is electrically connected to electrodes 426 through the one or more leads 424, and delivers each of the neurostimulation pulses through a set of electrodes selected from the electrodes 426. The stimulation output circuit 212 can implement, for example, the generating and delivery of a customized neurostimulation waveform (e.g., implemented from a parameter of a program selected with the closed-loop programming system) to an anatomical target of a patient.

The stimulation control circuit 514 represents an embodiment of the stimulation control circuit 214 and controls the delivery of the neurostimulation pulses using the plurality of stimulation parameters specifying the pattern of the neurostimulation pulses. In one embodiment, the stimulation control circuit 514 controls the delivery of the neurostimulation pulses using the one or more sensed physiological signals and processed input from patient feedback interfaces. The implant telemetry circuit 534 provides the implantable stimulator 421 with wireless communication with another device such as a device of the external system 410, including receiving values of the plurality of stimulation parameters from the external system 410. The implant storage device 532 stores values of the plurality of stimulation parameters, including parameters from one or more programs obtained using the program modeling and closed-loop programming techniques disclosed herein.

The power source 536 provides the implantable stimulator 421 with energy for its operation. In one embodiment, the power source 536 includes a battery. In one embodiment, the power source 536 includes a rechargeable battery and a battery charging circuit for charging the rechargeable battery. The implant telemetry circuit 534 may also function as a power receiver that receives power transmitted from external system 410 through an inductive couple.

In various embodiments, the sensing circuit 530, the stimulation output circuit 212, the stimulation control circuit 514, the implant telemetry circuit 534, the implant storage device 532, and the power source 536 are encapsulated in a hermetically sealed implantable housing. In various embodiments, the lead(s) 424 are implanted such that the electrodes 426 are placed on and/or around one or more targets to which the neurostimulation pulses are to be delivered, while the implantable stimulator 421 is subcutaneously implanted and connected to the lead(s) 424 at the time of implantation.

FIG. 6 illustrates an embodiment of a closed-loop programming system 602 used with an implantable neurostimulation system, such as the external system 410. Here, the closed-loop programming system 602 is illustrated to receive data (e.g., commands, parameters, program selections, information) directly or indirectly from a program modeling system or clinician/patient interaction computing device (not shown in FIG. 6, but discussed with reference to FIG. 7, below) to implement relevant closed-loop programming operations.

The closed-loop programming system 602 represents an embodiment of the programming device 302, and includes an external telemetry circuit 640, an external storage device 616, a programming control circuit 620, a user interface device 610, a controller 630, and an external communication device 618, to effect programming of a connected neurostimulation device. The closed-loop programming system 602 also includes a neurostimulation parameter generation circuit 660 to generate and select customized parameters for patient treatment, for implementation with programming via the connected neurostimulation device. The neurostimulation parameter generation circuit 660 is coupled to a model training circuit 662 used to train a programming model to the characteristics of a particular human patient, based on the context-dependent weighting techniques discussed herein. The neurostimulation parameter generation circuit 660 is also coupled to a model inference circuit 664 used to generate parameters or select programs, via the trained model, for the particular human patient.

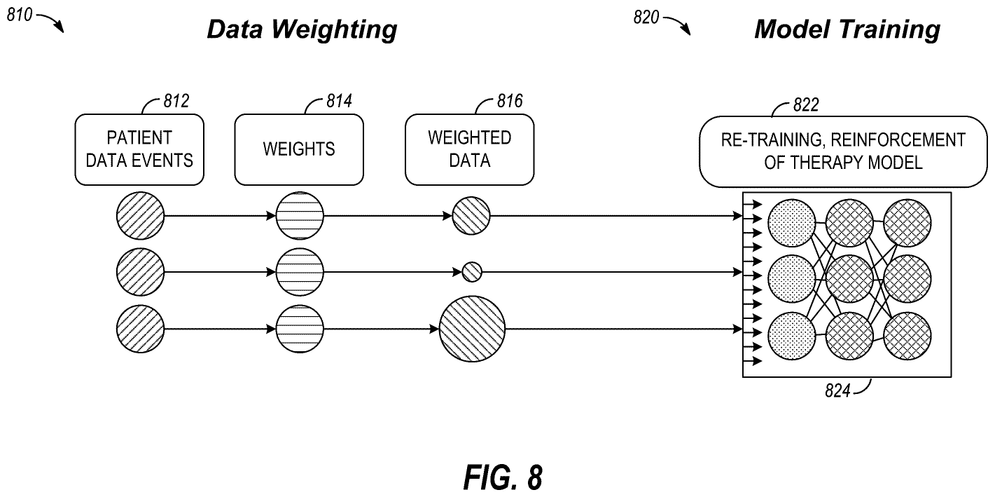

In specific examples, the model inference circuit 664 may implement logic that executes and operates a trained therapy model (e.g., an AI model to dynamically generate parameter output values or program output data, based on patient-specific data observations and historical events). This trained therapy model may be a programming model as discussed with reference to FIG. 7, below. The model training circuit 662 may implement logic to determine and apply weighting based on the type and duration of historical events and data observations to update (train) the therapy model, such as discussed with reference to FIGS. 8 to 10, below.

Thus, the operation of the neurostimulation parameter generation circuit 660, and specifically the configuration of a trained therapy model, is based on the operations of the data weighting and processing circuit 650. The data weighting and processing circuit 650 includes a patient input processing circuit 652 to collect and identify patient event information values and characteristics relevant to the patient state and the neurostimulation treatment. The patient input processing circuit 652 may collect information not directly related to the neurostimulation treatment or the medical condition being treated, to help identify confounding events. The data weighting and processing circuit 650 includes an event weighting circuit 654 to identify and calculate relevant weighting values for data collected during, after, or adjacent to respective historical events, such as discussed with reference to FIGS. 8 to 10, below.

The external telemetry circuit 640 provides the closed-loop programming system 602 with wireless communication to and from another controllable device such as the implantable stimulator 421 via the telemetry link 428, including transmitting one or a plurality of stimulation parameters (including selected, identified, or modified stimulation parameters of a selected program) to the implantable stimulator 421. In one embodiment, the external telemetry circuit 640 also transmits power to the implantable stimulator 421 through inductive coupling.

The external communication device 618 may provide a mechanism to conduct communications with a programming information source, such as a data service, program modeling system, to receive program information, models, weighting logic, functionality controls, or the like, via an external communication link (not shown). The external communication device 618 and the programming information source may communicate using any number of wired or wireless communication mechanisms described in this document, including but not limited to IEEE 802.11 (Wi-Fi), Bluetooth, Infrared, and like standardized and proprietary wireless communications implementations. Although the external telemetry circuit 640 and the external communication device 618 are depicted as separate components within the closed-loop programming system 602, the functionality of both of these components may be integrated into a single communication chipset, circuitry, or device.

The external storage device 616 stores a plurality of existing neurostimulation waveforms, including definable waveforms for use as a portion of the pattern of the neurostimulation pulses, settings and setting values, other portions of a program, and related treatment efficacy indication values. In various embodiments, each waveform of the plurality of individually definable waveforms includes one or more pulses of the neurostimulation pulses, and may include one or more other waveforms of the plurality of individually definable waveforms. Examples of such waveforms include pulses, pulse blocks, pulse trains, and train groupings, and programs. The existing waveforms stored in the external storage device 616 can be definable at least in part by one or more parameters including, but not limited to the following: amplitude, pulse width, frequency, duration(s), electrode configurations, total charge injected per unit time, cycling (e.g., on/off time), waveform shapes, spatial locations of waveform shapes, pulse shapes, number of phases, phase order, interphase time, charge balance, and ramping.

The external storage device 616 may also store a plurality of individually definable fields that may be implemented as part of a program. Each waveform of the plurality of individually definable waveforms is associated with one or more fields of the plurality of individually definable fields. Each field of the plurality of individually definable fields is defined by one or more electrodes of the plurality of electrodes through which a pulse of the neurostimulation pulses is delivered and a current distribution of the pulse over the one or more electrodes. A variety of settings in a program (including settings changed as a result of evaluation with the data weighting and processing circuit 650) may be correlated to the control of these waveforms and definable fields.

The programming control circuit 620 represents an embodiment of a programming control circuit 316 and may translate or generate the specific stimulation parameters or changes to be transmitted to the implantable stimulator 421, based on the results of the neurostimulation parameter generation circuit 660. The pattern may be defined using one or more waveforms selected from the plurality of individually definable waveforms (e.g., defined by a program) stored in an external storage device 616. In various embodiments, the programming control circuit 620 checks values of the plurality of stimulation parameters against safety rules to limit these values within constraints of the safety rules. In one embodiment, the safety rules are heuristic rules.

The user interface device 610 represents an embodiment of the user interface device 310 and allows the user (including a patient, caregiver, or clinician) to provide input relevant to therapy objectives, or to provide input relevant to the patient state. The user interface device 610 includes a display screen 612, a user input device 614, and may implement or couple to the data weighting and processing circuit 650. The display screen 612 may include any type of interactive or non-interactive screens, and the user input device 614 may include any type of user input devices that supports the various functions discussed in this document, such as a touchscreen, keyboard, keypad, touchpad, trackball, joystick, and mouse. The user interface device 610 may also allow the user to perform other functions where user interface input is suitable (e.g., to select, modify, enable, disable, activate, schedule, or otherwise define a program, sets of programs, provide feedback or input values, or perform other monitoring and programming tasks). Although not shown, the user interface device 610 may also generate a visualization of such characteristics of device implementation or programming, and receive and implement commands to implement or revert the program and the neurostimulator operational values (including a status of implementation for such operational values). These commands and visualization may be performed in a review and guidance mode, status mode, or in a real-time programming mode.

The controller 630 can be a microprocessor that communicates with the external telemetry circuit 640, the external communication device 618, the external storage device 616, the programming control circuit 620, and the user interface device 610, via a bidirectional data bus. The controller 630 can be implemented by other types of logic circuitry (e.g., discrete components or programmable logic arrays) using a state machine type of design. As used in this disclosure, the term “circuitry” should be taken to refer to either discrete logic circuitry, firmware, or to the programming of a microprocessor.

FIG. 7 illustrates, by way of example, an embodiment of data interactions among a closed-loop programming system 602, a program generation system 710, and clinician and patient interaction computing devices 730, 740, for operation of a neurostimulation device 750 based on context-dependent data weighting. At a high level, the closed-loop programming system 602 identifies and produces programming parameters 780 that are implemented to the neurostimulation device (e.g., using the programming techniques discussed above). The closed-loop programming system 602 produces these parameters through the use of a therapy model, such as an artificial intelligence model that evaluates closed-loop feedback from prior time periods and patient state data to generate or recommend new parameters. This therapy model is trained (and, re-trained or re-weighted) using context-dependent weighting of historical patient data that reduces the effect of certain events or data, to enable programming parameters to be customized to the patient and deliver an improved treatment via the neurostimulation device 750.

Specifically, the closed-loop programming system 602 operates program implementation logic 706 to generate programming parameters 780 in a closed loop fashion, based on the execution of one or more trained models, user data inputs (e.g., patient and clinician inputs related to whether the treatment is or is not working), sensor data inputs (e.g., physiological measurements related to whether the treatment is or is not working), and the like. The closed-loop programming system 602 may include a user interface 702 that allows control, modification, selection, or specification of data values and data types from an administrative user, a clinician, a patient, or the like.

The closed-loop programming system 602 may receive or access therapy models, programs, parameters, algorithms, logic, or other aspects from use of a program generation system 710. The program generation system 710 is shown in FIG. 7 in the form of a computing device (e.g., a server) with the computing device being specially programmed to communicate, over a network 720, various trained models 712 and program or parameter data 714 that are retrieved from a program and model data store 716. In an example, the training of the models 712 may be controlled by a healthcare provider, a device manufacturer, or another third party. The program generation system 710 may also implement selection logic 718 to respond to requests or inquiries for trained models, programs, parameter sets, such as requests from the closed-loop programming system 602. Other aspects of program settings, program modifications, constraints, rules, or like information related to programming or model operation may be communicated from the program generation system 710 to the closed-loop programming system 602. It will be understood that other form factors and embodiments of the program generation system 710, including in the integration of program modeling and selection logic into programming devices, data services, or information services, may also be provided.

The closed loop programming system 602 may perform training or modification of the trained models 712, in order to customize a particular model for use with a particular patient or patient condition. The training or modification may occur based on the use of logic including: time weighting logic 703 to apply weights to historical events or time-based data, based on the timing, duration, or amount of elapsed time since an event; intervention event weighting logic 704 to apply weights to historical events or time-based data, based on whether the event or data is identified as an intervention related to a medical condition being treated; and confounding event weighting logic 705 to apply weights to historical events or time-based data, based on whether the event or data is identified as a confounder (e.g., not related to and not caused by a medical condition being treated) and thus may provide an incorrect view of patient state or data measurements. The historical events or time-based data may be collected from data in user inputs provided by a patient or clinician, detected from sensor data, or provided from other third party sources (e.g., medical records). Finally, the closed loop programming system 602 may include programming implementation logic to invoke or use a trained model to generate the programming parameters 780 (or programs), which are then propagated to the neurostimulation device 750.

The patient interaction computing device 740 may include a computing device (e.g., personal computer, tablet, smartphone) or other form of user-interactive device that receives and provides interaction with a patient using a graphical user interface 742 and therapy feedback logic 744. Other form factors and interfaces such as smart speakers, audio interfaces, text interfaces, and the like may also be substituted for or augmented with the graphical user interface 742. The therapy feedback may be received via questionnaires, surveys, or selectable rating inputs, such as to collect input related to pain or satisfaction, or to identify a psychological or physiological state of the patient or treatment results. This feedback data may be used to classify outcomes and types of historical events, such as whether a particular patient state or outcome is (or is not) due to a confounding or intervention event. The clinician interaction computing device 730 may include a graphical user interface 732 and therapy feedback logic 734 with similar capabilities to the user interface 742 and therapy feedback logic 744, but adapted for use by a clinician (e.g., to provide enhanced functionality or features for physician control).

In an example, the closed-loop programming system 602 generates, selects, or communicates therapy suggestions 790 to the patient interaction computing device 740 based on recommended or indicated therapy objectives. These therapy suggestions 790 may include a recommendation or identification of the type of therapies to apply, or may include suggested therapy objective values. The therapy suggestions 790 may provide other instructions, recommendations, or feedback (including clinician recommendations, behavioral modifications, etc., selected for the patient). The therapy suggestions 790 may provide other relevant information (e.g., warnings, suggestions) based on the collection of sensor data 760 or other biopsychological/physiological state monitoring performed on the patient.

The closed-loop programming system 602 may also capture and evaluate sensor data 760 from one or more patient sensors 770 (e.g., wearables, sleep trackers, implantable devices, etc.) among one or more internal or external devices, including data occurring at the same time as intervention or confounding events. The sensor data 760 may be directly or indirectly used by the closed-loop programming system 602, to determine a customized and current state of the patient condition or treatment results, and to implement a recommended therapy based on a trained model. FIGS. 8 to 11 provide additional details on how context-dependent weights, based on certain types of historical events, are used to train or improve a model that will then evaluate this patient-specific sensor and state information for closed-loop programming.

In various examples, the neurostimulation device 750 includes sensors that contribute to the sensor data 760 evaluated by the closed-loop programming system. In an example, the patient sensors 770 are biopsychosocial sensors or physiological sensors that sense one or more biopsychosocial signals indicative of the biopsychosocial factors (e.g., stress and/or mood biomarkers) or physical factors. Examples of such sensors include a facial recognition sensor to sense the patient's facial expression, a voice sensor (e.g., microphone) to sense the patient's voice, a sleep sensor to sense the patient's sleep state (e.g., for detecting lack of sleep), a heart rate sensor to sense the patient's heart rate, a blood pressure sensor to sense the patient's blood pressure, an electrodermal activity (EDA) sensor to sense the patient's EDA (e.g., galvanic skin response), and/or an electrochemical sensor to sense stress biomarkers from the patient's body fluids (e.g., enzymes and/or ions, such as lactate or cortisol from saliva or sweat). Other types or form factors of sensor devices may also be utilized.

During a closed loop programming process, a variety of recommendations may be offered to a patient during a regular interval (e.g., daily). When a closed-loop programming system considers a longer review of patient events, the system will identify a variety of patient conditions caused by events as sicknesses, emotional distress or personal stress, and the like. In particular, certain events may be classified as “confounders” to an evaluation of neurostimulation data, such as a severe injury or device reprogramming, and therefore sensor or feedback data collected immediately after or during these events will not be useful for closed-loop programming. Likewise, older data and events may not be useful and not helpful for consideration in the closed-loop programming, if the patient state or the patient response to neurostimulation has changed.

Below, the approaches attempt to reduce the effect of confounders, interventions, and out-of-date patient data on a closed-loop therapy model. As noted above, the relevant data set used for closed-loop therapy could include free text, sensor data, or a variety of other data types or sources that could be polluted/corrupted by external events. Applying weighting formulas to patient data may help reduce the effects caused by interfering events such as confounding events or intervention events. The use of weighting may help also discount the effect of certain patient data evaluated for reprogramming, or help discount the effect of patient data collected from a particular period of time that is not relevant.

FIG. 8 illustrates, by way of example, an embodiment of data weighting operations 810 used for model training 820 of an artificial intelligence model, adapted for closed-loop operation of a neurostimulation device. Specifically, the data weighting operations 810 provides a high-level illustration of how patient data events 812 corresponding to patient historical events are associated with weights 814. This produces weighted data 816 for training of a model, as the importance or relevance of the data is increased or decreased by the weights. This weighted data 816 is then provided for re-training or reinforcement 822 of weights of a therapy model 824. (The therapy model 824 itself may be provided by a neural network or other data structure that uses various internal weights to guide inference or data processing operations).

In an example, application of the weights 814 involves applying a specific weighting equation, formula, or algorithm to decrease or increase the value of particular events (and the data associated with these events) that may interfere with the collection of data events evaluated in closed-loop neurostimulation. This weighting may be provided by a context-dependent weighting equation that incorporates temporal factors which affect the reliability of data (and thus, the relevance of this data for use in modifying, training, or adjusting a model). Some of the temporal factors that may be evaluated include:

Time elapsed (sigmoidal discount function).

Interventions related to the medical condition being treated. Examples of interventions include lead revision, additional surgery, or pain injections. Events that may be classified as an intervention are referred to herein as “intervention events.” In an example, intervention events are subject to a dual Heaviside function, which reduces (discounts) the impact/influence of data collected (if that data was collected during a time in which that function was in effect). The discount function that is used is a function of time, so the degree to which data is ‘discounted’ is described by that function shape over time.

Confounders that are not related to the medical condition being treated, such as new pain type since implant, disease progression/regression, death of a loved one or other personal event which could impact measures. Events that may be classified as confounders are referred to herein as “confounding events.” Confounding events also may be subject to a dual Heaviside function or another discount function. In this context, confounding events are those events that cause a change in one or more measured variables associated with a data evaluation model. Confounding events are typically not related to (and not caused by) the neurostimulation treatment, but may include events or conditions that are adjacent to the medical condition. For example, consider a scenario where a patient is being treated for neurostimulation for chronic back pain, but the patient pulls a back muscle that results in nociceptive pain that the neurostimulator is not intended to treat. The effects from this nociceptive pain may be characterized as resulting from a confounding event.

Confounding and intervention events include events that do not necessarily interfere with the neurostimulation treatment, but rather corrupt or reduce the reliability of data by adding additional sources of variance that are not addressable with the neurostimulation therapy. This corrupted data is less useful in training the model because the new source of variance of that data is addressable with the therapy. Thus, returning to the scenario where a patient pulls a back muscle, SCS treatment generally cannot improve the component of pain and events that are due to a pulled muscle. However, the region of the pain experienced with the pulled muscle is located in the lower back, and may overlap with the chronic neuropathic lower back pain that is addressable by SCS treatment.

In an example, previously captured events are combined into a weighting function that penalizes older data and penalizes time periods with unreliable data from interventions and confounders. This weighting functions may be represented by the following formula:

$$W ( t ) = elapsed ( t ) - interventions ( t ) - confounders ( t ) \qquad Equation 1$$

Where the following equation is used for determining elapsed time:

$$elapsed ( t ) = \frac{1}{1 + {e}^{- Slope * t - offset}} \qquad Equation 2$$

Here, slope represents the rate of reliability drop-off, and offset represents a maximum time of high data reliability. In further examples, the following values are used for interventions, and confounders:

$$interventions ( t ) = {\sum }_{events} \frac{discount\_factor}{1 + {e}^{- 2 * k * ( t - eventStart )}} - \frac{discount\_factor}{1 + {e}^{- 2 * k * ( t - eventStart - eventDuration )}} \qquad Equation 3$$

$$confounders ( t ) = {\sum }_{events} \frac{d i scount\_factor}{1 + {e}^{- 2 * k * ( t - e v e n t Start )}} - \frac{discount\_factor}{1 + {e}^{- 2 * k * ( t - eventStart - eventDuration )}} \qquad Equation 4$$

In Equations 3 and 4, t is time, and a larger k creates a sharper Heaviside analytic approximation; additionally, discount_factor is based on event classification and severity/impact of an event on data reliability.

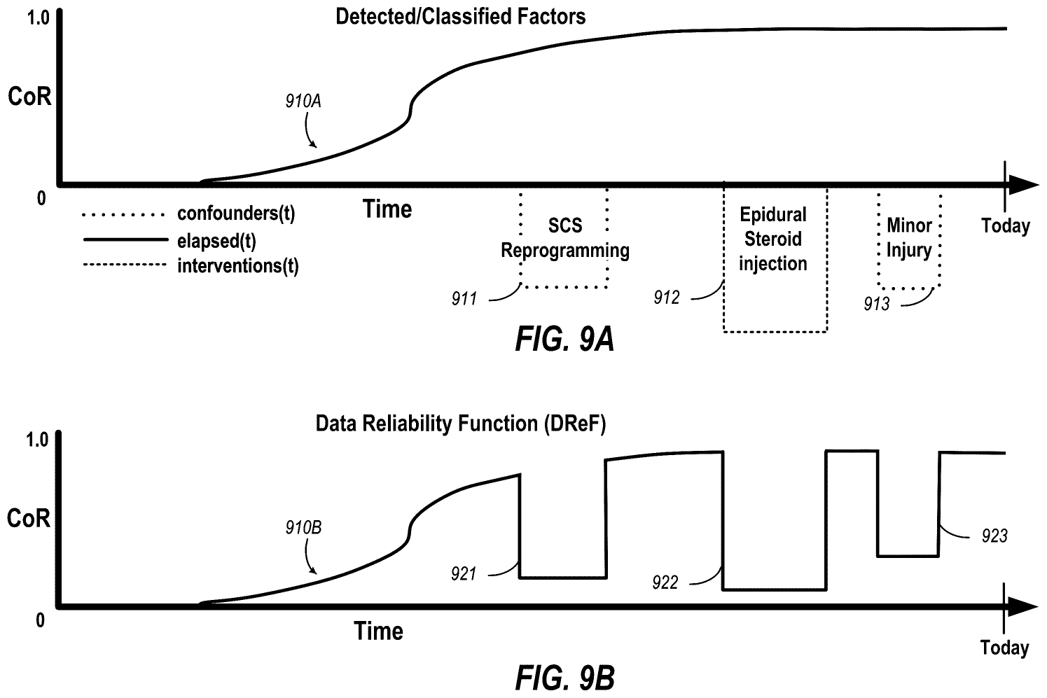

FIGS. 9A and 9B illustrates, by way of example, graphs that compare the coefficient of reliability (CoR) versus time, for various types of events. In these graphs, only three events are shown for purposes of illustration; it will be understood that many more events and types of events may be tracked and analyzed.

In FIG. 9A, the events that are identified include: a SCS reprogramming event 911 (occurring at the beginning of event 911, and having effects that last for some first duration of time); an epidural steroid injection event 912 (occurring at the beginning of event 912, and also having effects that last for a second duration of time); and a minor injury event 913 (occurring at the beginning of event 913, and having effects that last for a third duration of time). These events 911, 912, 913 are depicted relative to a graph 910A of time-based events during a prior time period, using a function that assigns a CoR value based on the amount of time that has elapsed. Here, the graph 910A of time-based events shows that a lower reliability value (e.g., representing lower relevance to model training or adaptation) is assigned for events and data observations that occur a long time ago, and an increased reliability value (e.g., representing higher relevance to model training or adaptation) is assigned for events and data observations that are more recent.

In FIG. 9A, each of the events 911, 912, 913 are classified from history data associated with the patient, and a duration (e.g., based on start time and end time) is determined for each classified event. Based on this analysis of the history data, it can be determined that the SCS reprogramming event 911 and the minor injury event 913 are confounding events, because these events do not provide a treatment of the medical condition being addressed with neurostimulation. Due to the confounding events, the patient data collected during or after these events may not be fully reliable to provide adjustments of closed-loop programming. The epidural steroid injection event 912, however, is an intervention event, because the occurrence of this event provides a treatment of the medical condition being addressed with neurostimulation. The patient data collected during this event also may not be fully reliable to provide adjustments of closed-loop programming, because the injection is likely to have interfered with (e.g., caused variance to) the patient data measurements collected during and after the injection.

In FIG. 9B, the graph 910B of the data reliability function is modified to show how the events 911, 912, 913 are subtracted to produce data adjustments 921, 922, 923, based on timing of the respective event, the respective duration of the event, and the significance of the event. The significance of the event (and, the amount of weighting applied to the event) may be determined from a classification or sub-type of the event, a severity or rating of the event, an amount of time that has passed since the event, and other characteristics of the event. Thus, this graph 910B shows how the value of all events is diminished over time, and how different types of events may be used to reduce the influence of data collected during the event periods.

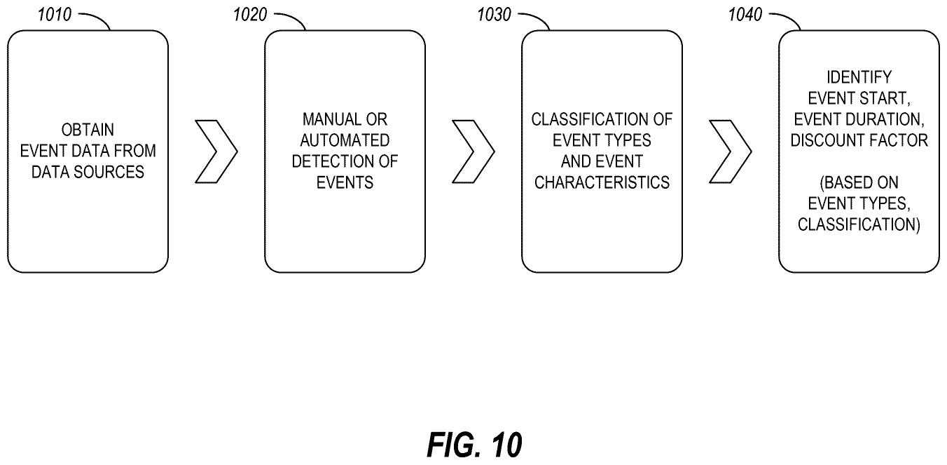

FIG. 10 depicts a data flow for detecting and analyzing events. Here, this data flow demonstrates a sequence for identifying relevant patient data and feedback information from historical data events, including the intervening and confounding event types discussed above.

At operation 1010, event data is obtained (e.g., extracted, queried, received) from one or more data sources. This may include the patient sensors 770, the neurostimulation device 750, feedback provided from the patient interaction computing device 740, feedback provided from the clinician interaction computing device 730, and the like. Other data sources may include processed medical data records, patient status or patient state data records, and the like.

At operation 1020, events that possibly interfere with patient data measurements (e.g., cause variance to the data measurements) are manually or automatically detected. Manual event detection may include the use of surveys or questionnaires to ask a patient whether certain life or health events have occurred, or the use of manually-applied or defined rules. Automatic event detection may include the application of certain patterns, rules, classifications, or regressions. An example of automatic event detection may include detecting that a patient was sick when the patient has certain physiological symptoms and visits a clinic or hospital. Various forms of artificial intelligence may be used for detection and classification of events.

At operation 1030, events are classified into one or more event types, and characteristics of the respective events are identified. These event types may include confounding events, intervention events, and regular health events (e.g., a classification for non-cofounding and non-intervention events). Confounding events (or intervention, regular health events) may include sub-types of events such as personal injury, emotional trauma, medication change, stimulator re-programming, etc. Other event types or taxonomies may be used. The characteristics of the events that may be identified or derived may include event timing (e.g., start, end, duration), event duration, event severity, event reliability or variability, and the like.

At operation 1040, individual events are classified and evaluated for use in a data weighting, such as with use of the weighting algorithms discussed above. The use of these algorithms may include the identification of an event start, event duration, and a discount factor (e.g., based on the event type or event characteristics). Additional characteristics or variations may be provided.

In a specific example, confounding event detection and classification may be used to identify confounders such as life events (e.g., not related to the medical condition being treated with neurostimulation) that could impact data relevance. Here, the data sources (e.g., evaluated in operation 1010) may include patient freeform text input from a patient smartphone app, a website portal, or another user interface. The detection of events (e.g., in operation 1020) may include the identification of particular keywords or answers that suggest some type of confounding event has occurred. The classification of events (e.g., in operation 1030) may involve a manual or automated evaluation of sub-types or categories such as injury (with available categories corresponding to minor, moderate, severe classifications), life event (with available categories corresponding to minor, moderate, severe classifications). The results of this detection (e.g., outputs produced by operation 1040) may include eventStart, eventDuration based on classification, discount_factor based on classification. The discount factor may correspond to the sub-type or category of the event. For example, a minor injury may be associated with a 3 day duration and a 0.2 discount_factor; a moderate injury may be associated with a 7 day duration and a 0.4 discount_factor; a severe injury may be associated with a 14 day duration and a 0.8 discount_factor.

In another specific example, intervention event detection and classification may be used to detect and classify medical interventions (related to the medical condition being treated with neurostimulation) that could impact data relevance. Here, the data sources (e.g., evaluated in operation 1010) may include patient freeform text from a patient smartphone app, a website portal, or another user interface, or a medical records data source from a medical provider. The detection of events (e.g., in operation 1020) may include the identification of particular keywords or medical codes (e.g., associated with a medical procedure at a doctor's office) or sensor/physiological data that suggests some type of intervention event has occurred. The classification of events (e.g., in operation 1030) may involve a manual or automated evaluation of sub-types or categories such as medical procedure (minor, moderate, major), neurostimulator reprogramming, neurostimulator lead revision (lead reposition, lead removal, lead addition, etc.). The results of this detection (e.g., outputs produced by operation 1040) may include eventStart, eventDuration based on classification, discount_factor based on classification. The discount factor may correspond to the sub-type or category of the event. For example, SCS reprogramming may be associated with a 7 day dropout with a 1.0 discount factor; SCS lead revision may be associated with a 14 day dropout with a 0.5 discount_factor.

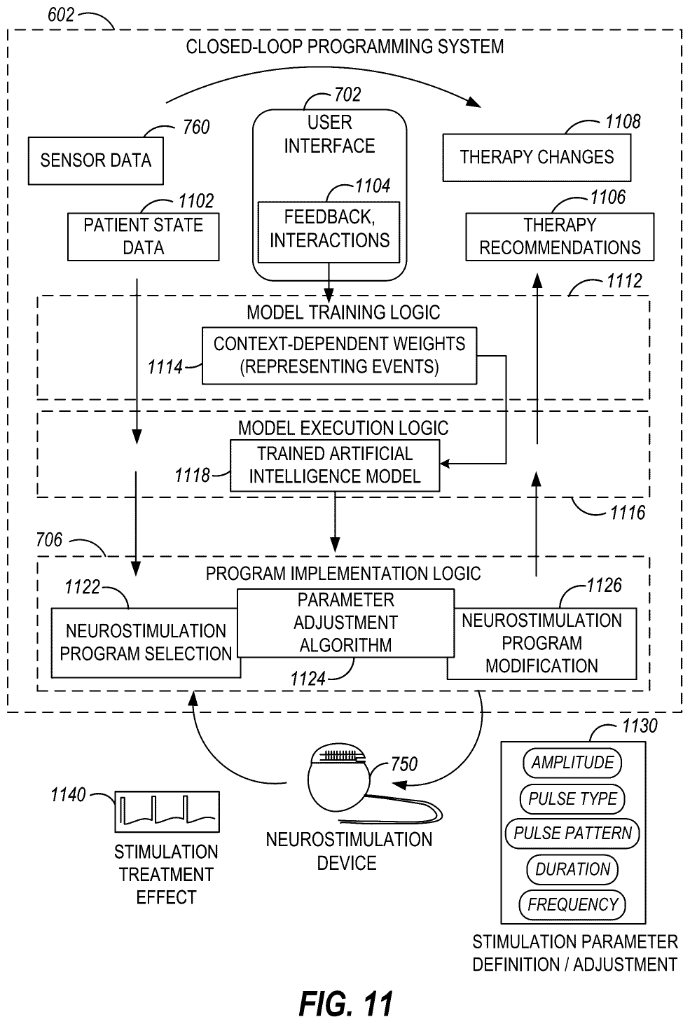

FIG. 11 illustrates, by way of example, an embodiment of a closed-loop processing flow for implementing neurostimulation treatment of a human patient, using context-dependent weighting for training data in a neurostimulation programming model. In addition to the sensor data 760 being processed by the closed-loop programming system 602, FIG. 11 also depicts the consideration of patient state data 1102 that may be derived from clinician or patient data inputs (e.g., medical records, patient survey or input answers, caregiver reports, etc.). The closed-loop programming system 602 is shown as providing parameter and program changes for the neurostimulation device 750 via one or more therapy changes 1108. Other outputs, such as one or more therapy recommendations 1106, or other forms of suggestions, commands, or information may be provided by the system 602.

The closed-loop programming system 602 is depicted as receiving feedback and interactions 1104 within its user interface 702, and the feedback and interactions 1104 are processed to identify context-dependent weights 1114 by the model training logic 1112. The context-dependent weights 1114 are applied to data used for training a therapy model, to produce a therapy model. An example of a therapy model includes a trained artificial intelligence model 1118 that is trained to produce (infer or generate) programming parameters for a neurostimulation device, based on input data such as history events unique to a particular patient. The trained artificial intelligence model 1118 is operated by model execution logic 1116 to produce parameters or other programming information from the trained artificial intelligence model 1118. These parameters are then provided to the program implementation logic 706.

The program implementation logic 706 may be implemented using a parameter adjustment algorithm 1124, which may be coordinated with neurostimulation program selection logic 1122 (e.g., to select a program with the identified parameters) or a neurostimulation program modification logic 1126 (e.g., to modify a program with the identified parameters). Some parameter changes may be implemented by a simple modification to a program operation; other parameter changes may require a new program to be deployed. The results of the parameter or program changes or selection causes a definition or adjustment of stimulation parameters 1130 at the neurostimulation device 750, causing a different or new stimulation treatment effect 1140. The use of this different or new stimulation treatment effect then can be monitored and recorded in event data (producing additional sensor data 760 or patient state data 1102), and a repeat of the closed-loop re-programming.

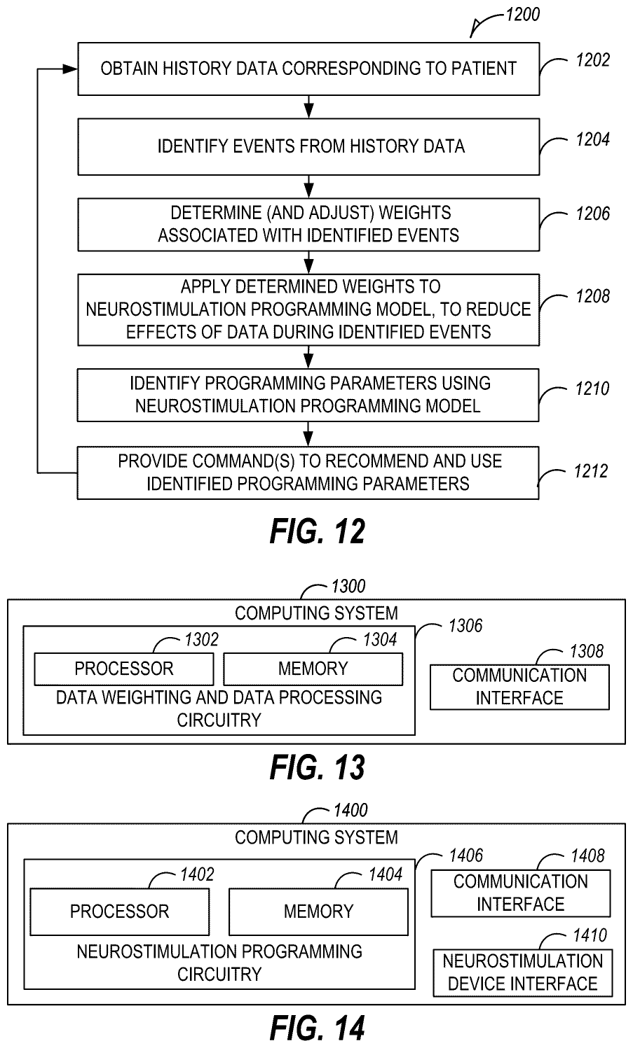

FIG. 12 illustrates, by way of example, an embodiment of a processing method 1200 implemented by a system or device for adapting a neurostimulation programming model used for neurostimulation treatment. For example, the processing method 1200 can be embodied by electronic operations performed by one or more computing systems or devices that are specially programmed to implement the event processing, model training, model execution, and neurostimulation programming functions described herein. In specific examples, the operations of the method 1200 may be implemented through the systems and data flows depicted above in FIGS. 6 to 11.

In an example, the method 1200 begins, at operation 1202, to obtain history data corresponding to a patient (e.g., a patient having a neurostimulation treatment delivered with a neurostimulation device to treat a medical condition, as discussed above). In an example, this history data is patient data observed during a prior time period, such as hours, days, months, etc. This history data may be extracted from a patient data source, such as a patient data source that provides at least one of: freeform text, voice recordings, survey data, or medical records data.

The method 1200 continues, at operation 1204, to identify events, including confounding and intervention events, from the history data. In various examples, the one or more events include an intervention event that is related to treatment of the medical condition of the human patient, and/or a confounding event that is not related to treatment of the medical condition of the human patient.

The method 1200 continues, at operation 1206, to determine and adjust weights of data associated with (e.g., collected during) the identified events, based on the type of events, and based on the timing and duration of the events. In an example applicable to an intervention event, this includes to calculate a weight that reduces the effects of the patient data throughout a duration of the intervention event. In an example applicable to a confounding event, this includes to calculate a weight that reduces the effects of the patient data on its impact in training or retraining a model, throughout a duration of the confounding event. For an AI model that is trained and/or continually refined using ongoing data, then in an ideal scenario the data that is collected for training or retraining the AI model would be only attributable to the therapy. If the data is due to other sources of variability—from intervention events or confounding events—then weights can be used to eliminate that data from influencing the training or retraining of the AI model (or, reduce the degree to which that data can influence the training or retraining).

In a further example, the weighting functions discussed above may be applied to determine and adjust the weights. For instance, this may include calculating weights for the patient data throughout the prior time period for respective measurements (observations) of the patient data, with a calculation of the weights that uses a discount to reduce effects of the respective measurements on the neurostimulation programming model, based on an amount of time elapsed. This may also include calculating the weights for the patient data based on a rate of decrease in data relevance and a maximum value of the data relevance. Further examples may also include calculating a weight based on a duration for the respective event, and calculating a weight based on an estimated impact of the respective event on data relevance, and/or calculating a weight based on an estimated impact that differs based on a type or severity of the respective event.

The method 1200 continues, at operation 1208, to apply the determined weights to data used in adapting or modifying the neurostimulation programming model, to reduce the effects of data collected during (e.g., associated with) the identified events. For instance, modifying a neurostimulation programming model may include performing a reinforcement or re-training of the neurostimulation programming model, using the weighted data. The neurostimulation programming model may be implemented as an artificial neural network or as a machine learning classifier, but other algorithms or types of models may also be used. One specific example of a neural network includes a neural network applying reinforcement learning using the techniques discussed herein, such as with a neural network implementing an algorithm that attempts to solve a contextual multi-armed bandit problem for reinforcement learning.

Further operations may include, at operation 1210, to identify programming parameters for use with the neurostimulation device, using the (trained) neurostimulation programming model. This may be followed by commands, at operation 1212, to provide commands, prompts, or other actions to recommend and use the identified programming parameters. The use of the identified programming parameters by the neurostimulation device may be used in a closed-loop process to obtain subsequent events (and repeating operations 1202-1210).

FIG. 13 illustrates, by way of example, a block diagram of an embodiment of a system 1300 (e.g., a computing system) implementing data weighting and data processing to modify operation of a closed-loop therapy model, such as the trained AI model 1118 discussed above. The system 1300 may be integrated or invoked as part of a remote control device, patient programmer device, clinician programmer device, program modeling system, or other external device, usable for the adjustment of neurostimulation programming with the programming model approaches discussed herein. In some examples, the system 1300 may be a networked device connected via a network (or combination of networks) to a programming device or programming service using a communication interface 1308. The network may include local, short-range, or long-range networks, such as Bluetooth, cellular, IEEE 802.11 (Wi-Fi), or other wired or wireless networks.

The system 1300 includes a processor 1302 and a memory 1304, which can be optionally included as part of data weighting and data processing circuitry 1306. The processor 1302 may be any single processor or group of processors that act cooperatively. The memory 1304 may be any type of memory, including volatile or non-volatile memory. The memory 1304 may include instructions, which when executed by the processor 1302, cause the processor 1302 to implement the features of the user interface, or to enable other features of the data weighting and data processing circuitry 1306. Thus, electronic operations in the system 1300 may be performed by the processor 1302 or the circuitry 1306.

For example, the processor 1302 or circuitry 1306 may implement any of the features of the method 1200 (including operations 1202, 1204, 1206, 1208) to obtain and process data, to produce weights, weighting results, or a weight-trained data model, based on the event data processing approaches discussed above. The system 1300 may save, communicate, or cause implementation of the weighting or training, directly or indirectly. It will be understood that the processor 1302 or circuitry 1306 may also implement other aspects of the logic and processing described above with reference to FIGS. 6-12, for use in a closed-loop system.

FIG. 14 illustrates, by way of example, a block diagram of an embodiment of a system 1400 (e.g., a computing system) implementing neurostimulation programming circuitry 1406 to cause programming of an implantable electrical neurostimulation device, for accomplishing the therapy objectives in a human subject as discussed herein. The system 1400 may be operated by a clinician, a patient, a caregiver, a medical facility, a research institution, a medical device manufacturer or distributor, and embodied in a number of different computing platforms. The system 1400 may be a remote control device, patient programmer device, program modeling system, or other external device, including a regulated device used to directly implement programming commands and modification with a neurostimulation device. In some examples, the system 1400 may be a networked device connected via a network (or combination of networks) to a computing system operating a user interface computing system using a communication interface 1408. The network may include local, short-range, or long-range networks, such as Bluetooth, cellular, IEEE 802.11 (Wi-Fi), or other wired or wireless networks.

The system 1400 includes a processor 1402 and a memory 1404, which can be optionally included as part of neurostimulation programming circuitry 1406. The processor 1402 may be any single processor or group of processors that act cooperatively. The memory 1404 may be any type of memory, including volatile or non-volatile memory. The memory 1404 may include instructions, which when executed by the processor 1402, cause the processor 1402 to implement the features of the neurostimulation programming circuitry 1406. Thus, the electronic operations in the system 1400 may be performed by the processor 1402 or the circuitry 1406.

The processor 1402 or circuitry 1406 may implement any of the features of the method 1200 (including operations 1210) that identify neurostimulation programming parameters from a trained (e.g., weight-adjusted) therapy model, and implement (e.g., save, persist, activate, control) the programming parameters or relevant programs in the neurostimulation device, with use of a neurostimulation device interface 1410. The processor 1402 or circuitry 1406 may further provide data and commands to assist the processing and implementation of the programming using communication interface 1408. It will be understood that the processor 1402 or circuitry 1406 may also implement other aspects of the programming devices and device interfaces described above with reference to FIGS. 6-11.

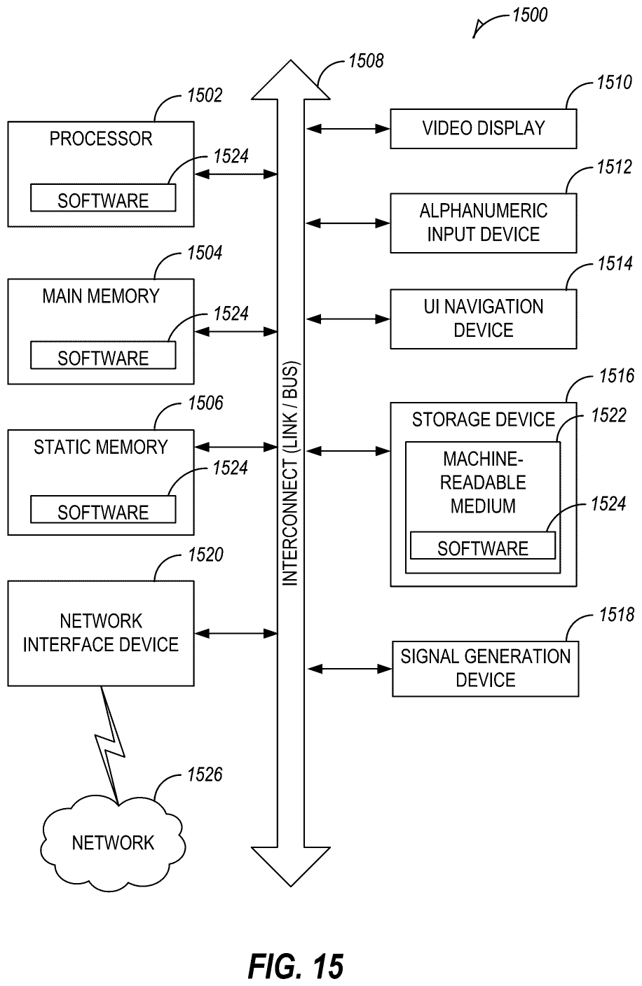

FIG. 15 is a block diagram illustrating a machine in the example form of a computer system 1500, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User's Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1500 includes at least one processor 1502 (e.g., a central processing unit (CPU), a graphics processing unit (GPU) or both, processor cores, compute nodes, etc.), a main memory 1504 and a static memory 1506, which communicate with each other via a link 1508 (e.g., bus). The computer system 1500 may further include a video display unit 1510, an alphanumeric input device 1512 (e.g., a keyboard), and a user interface (UI) navigation device 1514 (e.g., a mouse). In one embodiment, the video display unit 1510, input device 1512 and UI navigation device 1514 are incorporated into a touch screen display. The computer system 1500 may additionally include a storage device 1516 (e.g., a drive unit), a signal generation device 1518 (e.g., a speaker), a network interface device 1520, and one or more sensors (not shown), such as a global positioning system (GPS) sensor, compass, accelerometer, or other sensor. It will be understood that other forms of machines or apparatuses (such as PIG, RC, CP devices, and the like) that are capable of implementing the methodologies discussed in this disclosure may not incorporate or utilize every component depicted in FIG. 15 (such as a GPU, video display unit, keyboard, etc.).

The storage device 1516 includes a machine-readable medium 1522 on which is stored one or more sets of data structures and instructions 1524 (e.g., software) embodying or utilized by any one or more of the methodologies or functions described herein. The instructions 1524 may also reside, completely or at least partially, within the main memory 1504, static memory 1506, and/or within the processor 1502 during execution thereof by the computer system 1500, with the main memory 1504, static memory 1506, and the processor 1502 also constituting machine-readable media.

While the machine-readable medium 1522 is illustrated in an example embodiment to be a single medium, the term “machine-readable medium” may include a single medium or multiple media (e.g., a centralized or distributed database, and/or associated caches and servers) that store the one or more instructions 1524. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1524 may further be transmitted or received over a communications network 1526 using a transmission medium via the network interface device 1520 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

The above detailed description is intended to be illustrative, and not restrictive. The scope of the disclosure should, therefore, be determined with references to the appended claims, along with the full scope of equivalents to which such claims are entitled.

What is claimed is:

1. A device for adapting a neurostimulation programming model used for neurostimulation treatment, the device comprising:
at least one processor and at least one memory;
event data processing circuitry, operably coupled with the processor and the memory, the event data processing circuitry configured to:
obtain patient data observed during a prior time period, the patient data for a human patient having a neurostimulation treatment delivered with a neurostimulation device to treat a medical condition;
identify one or more events experienced by the human patient during the prior time period that cause variance in measurements of the patient data; and
determine weighted data by weighting the patient data observed during the one or more events; and
neurostimulation programming circuitry, operably coupled with the at least one processor and the at least one memory, the neurostimulation programming circuitry configured to:
modify the neurostimulation programming model based on the weighted data, the neurostimulation programming model to generate updated programming parameters for the neurostimulation treatment, wherein the weighting of the patient data reduces effects of the patient data during the one or more events on the neurostimulation programming model.

2. The device of claim 1, wherein the one or more events include an intervention event that is related to treatment of the medical condition of the human patient, and wherein to determine the weighted data for the intervention event includes to calculate a weight that reduces the effects of the patient data throughout a duration of the intervention event.

3. The device of claim 1, wherein the one or more events include a confounding event that is not related to treatment of the medical condition of the human patient, and wherein to determine the weighted data for the confounding event includes to calculate a weight that reduces the effects of the patient data throughout a duration of the confounding event.

4. The device of claim 1, the event data processing circuitry further configured to:
calculate weights for the patient data throughout the prior time period for respective measurements of the patient data, wherein the calculation of the weights uses a discount that reduces effects of the respective measurements on the neurostimulation programming model, based on an amount of time elapsed.

5. The device of claim 4, wherein to calculate the weights for the patient data throughout the prior time period for the respective measurements of the patient data, includes to calculate the weights for the patient data based on a rate of decrease in data relevance and a maximum value of the data relevance.

6. The device of claim 1, wherein to determine the weighted data corresponding to a respective event of the one or more events, includes to calculate a weight based on a duration for the respective event.

7. The device of claim 1, wherein to determine the weighted data corresponding to a respective event of the one or more events, includes to calculate a weight based on an estimated impact of the respective event on data relevance, and wherein the estimated impact differs based on a type or severity of the respective event.

8. The device of claim 1, wherein to modify the neurostimulation programming model includes to perform reinforcement or re-training of the neurostimulation programming model using the weighted data, and wherein the neurostimulation programming model is implemented as an artificial neural network or as a machine learning classifier.

9. The device of claim 1, the neurostimulation programming circuitry further configured to:
identify, with the use of the neurostimulation programming model, programming parameters for use with the neurostimulation device; and
communicate, to the neurostimulation device, at least one command to cause the use of the identified programming parameters.

10. The device of claim 9, wherein the identified programming parameters specify operation of a neurostimulation program including one or more of: pulse patterns, pulse shapes, a spatial location of pulses, waveform shapes, or a spatial location of waveform shapes, for modulated energy provided with a plurality of leads of the neurostimulation device.

11. A method for use to adapt a neurostimulation programming model used for neurostimulation treatment, the method comprising a plurality of operations executed with at least one processor of a computing device, the plurality of operations comprising:
obtaining patient data observed during a prior time period, the patient data for a human patient having a neurostimulation treatment delivered with a neurostimulation device to treat a medical condition;
identifying one or more events experienced by the human patient during the prior time period that cause variance in measurements of the patient data;
determining weighted data by weighting the patient data observed during the one or more events; and
modifying the neurostimulation programming model based on the weighted data, the neurostimulation programming model to generate updated programming parameters for the neurostimulation treatment, wherein the weighting of the patient data reduces effects of the patient data during the one or more events on the neurostimulation programming model.

12. The method of claim 11, wherein the one or more events include an intervention event that is related to treatment of the medical condition of the human patient, and wherein to determine the weighted data for the intervention event includes to calculate a weight that reduces the effects of the patient data throughout a duration of the intervention event.

13. The method of claim 11, wherein the one or more events include a confounding event that is not related to treatment of the medical condition of the human patient, and wherein to determine the weighted data for the confounding event includes to calculate a weight that reduces the effects of the patient data throughout a duration of the confounding event.

14. The method of claim 11, the operations further comprising:
calculating weights for the patient data throughout the prior time period for respective measurements of the patient data, wherein the calculation of the weights uses a discount that reduces effects of the respective measurements on the neurostimulation programming model, based on an amount of time elapsed.

15. The method of claim 14, wherein calculating the weights for the patient data throughout the prior time period for the respective measurements of the patient data, includes calculating the weights for the patient data based on a rate of decrease in data relevance and a maximum value of the data relevance.

16. The method of claim 11, wherein determining the weighted data corresponding to a respective event of the one or more events, includes calculating a weight based on a duration for the respective event.

17. The method of claim 11, wherein determining the weighted data corresponding to a respective event of the one or more events, includes calculating a weight based on an estimated impact of the respective event on data relevance, and wherein the estimated impact differs based on a type or severity of the respective event.

18. The method of claim 11, wherein modifying the neurostimulation programming model includes performing reinforcement or re-training of the neurostimulation programming model using the weighted data, and wherein the neurostimulation programming model is implemented as an artificial neural network or as a machine learning classifier.

19. The method of claim 11, the operations further comprising:
identifying, with the use of the neurostimulation programming model, programming parameters for use with the neurostimulation device; and
communicating, to the neurostimulation device, at least one command to cause the use of the identified programming parameters.

20. The method of claim 19, wherein the identified programming parameters specify operation of a neurostimulation program including one or more of: pulse patterns, pulse shapes, a spatial location of pulses, waveform shapes, or a spatial location of waveform shapes, for modulated energy provided with a plurality of leads of the neurostimulation device.
